(19) United States
(12) Patent Application Publication
(10) Pub. No.: US 2005/0032121 A1
(43) Pub. Date: Feb. 10, 2005

(54) Method of analyzing data obtained from a biological assay

(75) Inventor: S. Thomas King-Spengler, Rockford, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Pierce Biotechnology, Inc., Rockford, IL (US)

(21) Appl. No.: 10/898,920

(22) Filed: Jul. 26, 2004

Related U.S. Application Data
(60) Provisional application No. 60/492,514, filed on Aug. 4, 2003.

(51) Int. Cl. G01N 33/53; G01N 33/567; G06F 19/00; G01N 33/48; G01N 33/50

(52) U.S. Cl. 435/7.1; 702/19

(57) ABSTRACT

A method of analyzing data obtained from a biological assay, which method comprises (a) performing a biological assay, which quantitates a biological activity, (b) obtaining data resulting from the biological assay, and (c) analyzing the data using a dimensionally homogeneous equation, whereupon the data obtained from the biological assay are analyzed.

### FIELD OF THE INVENTION

This invention pertains to a method of analyzing data obtained from a biological assay.

### BACKGROUND OF THE INVENTION

The growth in pharmaceutical libraries has motivated laboratories to design ultra-high throughput, miniaturized methods of screening compounds for potential drug candidates (Lavery et al., J. Biomol. Screen 6(1), 3-9 (2001); Garyantes, Drug Disc. Today 7 (9), 489-490 (2002); Berg et al., Biomol. Screen. 6 (1), 47-56 (2001); Aviezer et al., J. Biomol. Screen. 6 (3), 171-177 (2001); Enomoto, J. Biomol. Screen. 5 (4), 263-268 (2000); Menke, J. Auto Met. And Manag. Chem. 22 (5), 143-144 (2000); Rudiger et al., J. Biomol. Screen. 6 (1), 29-37 (2001); and Entzeroth, J. Auto Met. And Manag. Chem. 22 (6), 171-173 (2000)). Ultra high throughput screening (uHTS), which is defined as screening more than 100,000 assays/day in a maximum sample volume of 10 μl, allows the cost and time of screening the many compounds of these large libraries to be decreased (Lavery et al., J. Biomol. Screen 6(1), 3-9 (2001); Turconi et al., J. Biomol. Screen. 6 (5), 275-290 (2001); and Zhang et al., J. Biomol. Screen. 4 (2), 67-73 (1999)). Operations, as a result, have become increasingly automated and integrated with microfluidic nanoscale methods to decrease reagent cost and to increase throughput. Consequently, applications based on a 96-well plate format have moved to a format involving 384-well plates, which will soon move to a 1536-well plate format. The increase in sample throughput brought on by the growth in pharmaceutical libraries is accommodated by the use of such homogeneous low-volume formats.

The complexity of drug screening assays is increased, not only by the high number of compounds found within each library, but also by the fact that each of the drug candidates affect their target differently. For instance, a pharmaceutical library containing inhibitors of a particular enzyme can contain inhibitors that inhibit the function of the enzyme in very different ways. In particular, the library can contain both competitive inhibitors and non-competitive inhibitors, each class of inhibitors affecting the enzyme velocity differently from the other. Also, inhibitors having different target active sites also affect the enzyme velocity in different ways (Kuo, C Oxford: New York (1994); Bates et al., J. Biol. Chem. 275 (15), 10968-10975 (2000); Izzard et al., Cancer Res. 59, 2581-2586 (1999); Shawver et al., Cancer Cell 1, 117-123 (2002); Blume-Jensen et al., Nature 411, 355-365 (2001); Newton, J. Biol. Chem. 270 (48), 28495-28498 (1995); Wisniewski, Cancer Res. 62, 4244-4255 (2002); Sawyers, Cancer Cell 1, 413-415 (2002); Nishizuka, Science 258, 607-612 (1992); Dekker et al., Science 19, 73-77 (1994); Bell, J. Biol. Chem. 266 (8), 4661-4664 (1991); Nishizuka, Science 258 (5082), 607-614. (1992); Lefebvre et al., J. Cell. Biochem. 75: 272-287 (1999); Silverstein et al., PNAS, 99 (7), 4221-4226 (2002); Hess et al., Nature Gen., 32, 201-205 (2002); Nagar et al., Cancer Res. 62, 4236-4243 (2002); Hall et al., Blood 98 (7) 2014-2021 (2001); and Schechtman et al., Met. Enzymol., 345 (37) 470-487 (2002).

In view of the foregoing, it has become increasingly difficult to analyze data obtained from biological assays, which quantitate a biological activity, due to the different formats of the assay (i.e., 96-well plate format vs. 384-well plate format vs. 1536-well format) and due to the differences in the ways in which the drug candidates of the pharmaceutical libraries interact with and act on the drug target. Therefore, there is a need in the art for a method of analyzing data obtained from such complex biological assays, such that the identification of the best drug candidate out of the library of candidates can easily be achieved.

The present invention provides such a method of analyzing data obtained from a biological assay. This and other objects and advantages of the invention, as well as additional inventive features, will be apparent from the description of the invention provided herein.

### BRIEF SUMMARY OF THE INVENTION

The invention provides a method of analyzing data obtained from a biological assay, which method comprises (a) performing a biological assay, which quantitates a biological activity, (b) obtaining data resulting from the biological assay, and (c) analyzing the data using a dimensionally homogeneous equation. Upon this method, the data obtained from the biological assay are analyzed.

### BRIEF DESCRIPTION OF THE DRAWINGS

FIG. 1 is a graph of relative fluorescence units (RFU) vs. concentration of Protein Kinase C (PKC; mU/μl of reaction), showing data obtained from different plate formats with identical reaction mixtures in a PKC assay using IQ™ Platform (Pierce Biotechnology, Inc., Rockford, Ill.). Pseudosubstrate labeled with rhodamine was used as the substrate.

FIG. 2 is a graph of non-dimensionalized fluorescence (ρ) vs. concentration of PCK (mU/μl of reaction (rxn)) for all three well formats. For each well format, the curves are superimposable.

FIG. 3 is a graph of RFU vs. concentration of inhibitor (nm) growing raw data of inhibition curves of various classes of PKC inhibitors. Difference in baseline RFU's are dependent on effects of enzyme velocity based on inhibitor class. Inhibitor class is shown in table 1.

FIG. 4 is a graph of Pᵢ vs. concentration of inhibitor (nm) showing dose response inhibition curves upon transformation of the raw RFU into dimensional group ρ. Different potencies of various inhibitors are seen. Also, differential Hill coefficients affect the slope of the intermediate region.

FIG. 5 is a graph of Pᵢ vs. ψᵢ showing a simulation of non-dimensionalized dose response inhibition curves with different Hill slopes.

FIG. 6 is a graph of Pᵢ vs. ψᵢ showing a non-dimensionalized dose response inhibition curve from inhibition data. Varying Hill slopes represent different classes of inhibitors having a different effect in binding cooperativity.

FIG. 7 is a graph of concentration of bound ligand (nM) vs. concentration of ligand (nM) showing a simulation of ligand binding assay.

FIG. 8 is a graph of ρ vs. concentration of ligand (nM) showing a ligand binding assay normalized to the average number of available binding sites.

FIG. 9 is a graph of ρ vs. concentration of ligand (nM) showing a ligand binding assay normalized to the average number of available binding sites for receptor molecules with different subsequent binding affinities.

### DETAILED DESCRIPTION OF THE INVENTION

Traditionally, engineering questions regarding fluid flow, heat flow, and diffusion could not be answered theoretically due to their complex nature and number of variables that affect their behavior. In these cases, no mathematical equation could be modeled to match observed phenomena without empirical data. For example, the thermal conductivity of a metal for a long, smooth, flat, straight, cooling fin depends on many variables including the length and diameter of the fin, the steady state temperature of the fin, the material of the fin, and others. If any one of these variables is changed, the effective heat dispersed by the fin also changes. Determining the dependence of all of these variables on the heat dispersed would be very costly. In other words, an entire plant at this scale would have to be dedicated for research, instead of production. One way to design experiments to find the optimal conditions for a desired application at large scale would be to build a miniaturized pilot scale model of the desired application (McCabe et al. Unit Operations of Chemical Engineering McGraw-Hill: St. Louis (1993); Bird et al., Transport Phenomena Wiley, New York. (1960); Deen, Analysis of Transport Phenomena, Oxford: New York. (1998); and Bridgman, Dimensional Analysis, AMS Press: New York (1978)). This pilot scale model plant would be scaled such that every piece of equipment, whether it is a pipe, cooling fin; etc., would be proportional to a constant value. As such, it would be the identical plant, only on a smaller magnitude.

Dimensional analysis is referred to as a method that lies between formal mathematical theory and empirical data. Dimensional analysis suggests that an equation must be dimensionally homogeneous and proportional to scale. Therefore, it is possible to group factors into dimensionless variables that are independent of magnitude. In essence, an experiment at large scale would have identical dimensionless values as one that was preformed at pilot scale (McCabe et al. Unit Operations of Chemical Engineering McGraw-Hill: St. Louis (1993); Bird et al., Transport Phenomena Wiley, New York. (1960); Deen, Analysis of Transport Phenomena, Oxford: New York. (1998); and Bridgman, Dimensional Analysis, AMS Press: New York (1978)).

The present invention provides a method, which applies dimensional analysis to problems of the biological field. In this regard, the present invention provides a method of analyzing data obtained from a biological assay, which method comprises (a) performing a biological assay, which quantitates a biological activity, (b) obtaining data resulting from the biological assay, and (c) analyzing the data using a dimensionally homogeneous equation. Using this method, the data obtained from the biological assay are analyzed.

For purposes of the present inventive method, the biological assay can be any biological assay, provided that it is an assay that quantitates a biological activity. The biological activity can be quantitated through indirect means. For instance, a signal that quantitatively correlates with the biological activity can be measured or quantitated in the assay, in order to quantitate the biological activity. The signal can be any detectable and quantifiable signal, such as a fluorescent signal, a colorimetric signal, a radioactive signal, or the quenching of any of the foregoing.

The biological activity quantitated by the biological assay can be, for example, the activity of one or more enzymes. The term “enzyme” as used herein refers to any of numerous proteins or conjugated proteins produced by living organisms and functioning as biochemical catalysts. The living organism can be any living organism, such as a prokaryote or a eukaryote, e.g., a bacterium, a virus, a yeast, a fungus, an algae, a plant, a bird, a reptile, or a mammal. The reaction that is catalyzed by the enzyme can take place inside or outside of a cell. The enzyme can be, but is not limited to, a kinase, a phosphatase, a hydrase, a lyase, a dehydrogenase, a transcription factor, a GTPase, a protease, a ubiquitinase, a transferase, a polymerase, a transcriptase, a peroxidase, a nuclease, a caspase, or an acetylase.

The biological activity that is quantitated by the biological assay can alternatively be the activity of one or more enzyme inhibitors, or the biological activity can be the activity of one or more enzyme inducers. The term “enzyme inhibitor” as used herein refers to any agent that prevents an enzyme from carrying out its catalytic activity. The enzyme inhibitor can be a reversible inhibitor, i.e., one that rapidly dissociates from the enzyme, or an irreversible inhibitor, i.e., one that slowly dissociates from the enzyme. The enzyme inhibitor can be a competitive inhibitor, i.e., a reversible inhibitor that binds to the enzyme at the active site and diminishes the rate of catalysis by reducing the proportion of enzyme molecules bound to a substrate, or a non-competitive inhibitor, i.e., a reversible inhibitor that binds to the enzyme, but does not prevent the substrate from binding. In non-competitive inhibition, the inhibitor acts by decreasing the turnover number, rather than by diminishing the proportion of enzyme molecules that are bound to substrate. The enzyme inhibitor can be an uncompetitive inhibitor, i.e., a reversible inhibitor that binds to the enzyme only when the substrate is bound to the enzyme. Like a non-competitive inhibitor, an uncompetitive inhibitor binds to the enzyme at a site removed from the active site. However, the uncompetitive inhibitor can only bind to the enzyme when it is in the form of the enzyme substrate complex. Once the uncompetitive inhibitor is bound, the reaction is not catalyzed (Stryer, Biochemistry, 4^{th} ed., W.H. Freeman and Co., New York, N.Y., (1995); and Lenninger et al., Principals of Biochemistry 2^{nd} Ed., Worth Publishing Inc., New York N.Y. (1993)). The term “enzyme inducer” as used herein means an agent that induces, stimulates, or promotes the catalytically active state of an enzyme. The enzyme inducer can stimulate the catalytically active state of an enzyme by any number of means, such as by changing its conformational state, bringing it closer to its substrate, releasing an inhibitory protein; etc.

The biological activity quantitated by the biological assay can be a ligand binding to a target. The term “ligand” as used herein refers to any biological, chemical, or biochemical agent, such as a compound, molecule, or cell that binds to another biological, chemical, or biochemical agent, i.e., its target. The ligand can be isolated from natural or synthetically produced materials. The ligand can be endogenous or exogenous to a prokaryote or eukaryote or archea, e.g., bacteria, a fungus, yeast, plant, or a mammal. Suitable ligands include, but are not limited to, cells, bacteria, viruses, yeast, proteins, peptides, amino acids, nucleic acids, carbohydrates, lipids, drugs, synthetic inorganic compounds, synthetic organic compounds, antibody preparations (e.g., antibody fragments, chemically-modified antibodies, and the like), sugars, isoforms of any of the foregoing, and combinations of any of the foregoing.

Organic molecules include, for example, synthetic organic compounds typically employed as pharmacotherapeutic agents. Such molecules are, optionally, mass-produced by combinatorial synthetic methods or, more specifically, by strategic syntheses devised to arrive at specific molecules. Likewise, organic molecules also include natural products and analogues, whether extracted from their natural environment or strategically synthesized. The term “organic” as used herein is not intended to be limited to molecules comprised only of carbon and hydrogen, but rather is used in its broader sense as encompassing macromolecules of biological origin.

The term “peptide” as used herein refers to an entity comprising at least one peptide bond, and can comprise either D and/or L amino acids. The ligand can be a peptide consisting essentially of about 2 to about 10 amino acids (e.g., about 2, 3, 4, 5, 6, 7, 8, 9, or 10 amino acids). The peptide ligands can be generated by combinatorial approaches, i.e., techniques commonly employed in the generation of a combinatorial library, e.g., the split, couple, recombine method or other approaches known in the art (see, e.g., Furka et al., Int. J. Peptide Protein Res., 37: 487-493 (1991); Lam et al., Nature, 354: 82-84 (1991); International Patent Application WO 92/00091; and U.S. Pat. Nos. 5,010,175, 5,133,866, and 5,498,538). The expression of peptide libraries also is described in Devlin et al., Science, 249: 404-406 (1990). In peptide libraries, the number of discrete peptides of differing sequence increases dramatically with the number of coupling reactions performed, the size of the peptide, and the number of distinct amino acids utilized. For example, the random incorporation of 19 amino acids into pentapeptides produces up to 2,476,099 (19⁵) individual peptides of differing sequence (Lam et al., supra). Combinatorial methods allow generation of libraries of ligands directly on a support. Typically, the ligands are synthesized on particles of support media, such that multiple copies of a single ligand are synthesized on each particle (e.g., bead), although this is not required in the context of the invention.

The biological activity quantitated by the biological assay can alternatively be the activity of a ligand inhibitor or the activity of a ligand inducer, wherein the term “ligand inhibitor” as used herein refers to an agent that inhibits the ligand from binding to its target and the term “ligand inducer” as used herein refers to an agent that induces, stimulates, promotes, or enhances the ligand binding to its target.

As used herein, the terms “inhibitor” and “inducer” do not necessarily imply a complete inhibition or induction. Rather, there are varying degrees of inhibition and of induction of which one of ordinary skill in the art recognizes as having a potential benefit or therapeutic effect. In this regard, the enzyme inhibitor can inhibit the enzyme to any extent, and the enzyme inducer can induce the enzyme to any extent. Furthermore, in this respect, the ligand inhibitor can inhibit the ligand from binding to its target to any extent, while the ligand inducer can promote or enhance ligand binding to any extent.

Methods of performing such biological assays suitable for the present inventive methods, as well as methods of obtaining the data therefrom, are known in the art. See, for instance, Kuo, Protein Kinase C. Oxford, New York N.Y., (1994); Reith, Protein Kinase Protocols. Humana Press, Totowa N.J. (2001); Huijing et al., Protein Phosphorylation in Control Mechanisms. Academic Press, New York N.Y. (1973); Hardie et al., The Protein Kinase Facts Book. Academic Press, San Diego Calif. (1995); and Example 1 set forth below for kinase assays. See, for example, Latchman, Transcription Factors: A Practical Approach, Oxford, New York N.Y. (1999); Ausubel et al., Short Protocols in Molecular Biology. Wiley, New York N.Y. (1999); Sambrook et al., Molecular cloning: a laboratory manual. Cold Spring Harbor, Plainview N.Y. (1989); Goodbourn, Eukaryotic Gene Transcription. Oxford, New York N.Y. (1996); McKnight et al., Transcriptional Regulation. Cold Spring Harbor, Plainview N.Y. (1992); and Gluzman, Eukaryotic Transcription. Cold Spring Harbor, Plainview N.Y. (1985) for transciption factor assays. See, for instance, Ludlow, J. W. Protein Phosphatase Protocols. Humana Press, Totowa N.J. (1998); Martin et al., Chacterization and Assay of Phosphatidate Phosphatase, Methods Enzymol. 197, 553-563 (1991); McComb et al. Alkaline Phosphatase. Plenum Press, New York N.Y. (1979); Manson, Immunochemical Protocols, Methods in Molecular Biology. Humana, Totowa N.J. (1992); Hamburger, Biochem Biophys Acta 1157 (1): 93-101 (1993); and Zhang, et al., Anal. Biochem 211 (1): 7-15 (1993), for phosphatase assays. Winzor Quantitative Characterization of Ligand Binding. Wiley-Liss, New York N.Y. (1995); Cass et al., Immobilized Biomolecules in Analysis: A Practical Approach. Oxford, New York N.Y. (1998); Hulme, Receptor-Ligand Interactions: A Practical Approach. Oxford New York N.Y. (1992); Burgen, Receptor Subunits and Complexes. Cambridge New York N.Y. (1992); Keen Receptor Binding Techniques, Methods in Molecular Biology. Humana Press, Totowa N.J. (1999); Boulton, Receptor Binding. Humana Press, Clifton N.J. (1986); O'Brien, Clinical Pharmacology, New York N.Y. (1986); Englebienne, Immune and Receptor Assays in Theory and Practice. CRC Press, Boca Raton Fla. (2000), and Example 2 set forth below for ligand binding assays. See, for instance, Hawcroft, Quantitative Bioassay. Chichester, New York N.Y. (1987); Finney, Statistical Method in Biological Assays. Hafner, New York N.Y. (1964); Hewitt, Microbiological Assay: An introduction to Quantitative Principals and Evaluation Academic Press, New York N.Y. (1977); Savage et al., Avadin Biotin Chemistry: A Handbook, Pierce, Rockford Ill. (1991); and Much, Bioassays: A Handbook of Quantitative Pharmacology (1931). Williams and Wilkins, Baltimore Md. for general bioassay design and statistical analysis.

In the present inventive method, the data obtained from the biological assay are analyzed using a dimensionally homogeneous equation. The term “dimensionally homogeneous equation” as used herein refers to a mathematical relationship that describes a natural phenomenon with dimensionless variables that are independent of magnitude and are proportional to scale. A dimensionally homogeneous equation suitable for use in the present inventive method allows one of ordinary skill in the art to analyze data obtained from a biological assay of a particular scale and apply it to another scale. In this regard, the dimensionally homogeneous equation allows for one to perform a biological assay utilizing a 96-well plate and apply the data obtained from this scale to a larger scale, such as a 384-well plate or a 1536-well plate. A dimensionally homogeneous equation also allows one to analyze the data obtained on one day with one set of reagents and/or instruments and compare the data to another set of data obtained on a different day and/or obtained with a different set of reagents and/or instruments. Furthermore, a dimensionally homogeneous equation allows one of ordinary skill in the art to compare directly the data obtained with different types of enzyme inhibitors or enzyme inducers, for example, despite the way in which the inhibitors or inducers act on the enzyme.

The dimensionally homogeneous equation used in the present inventive method depends on the biological activity being quantitated by the biological assay, i.e., depends on the type of biological assay. For instance, if the biological activity is the activity of one or more enzyme inhibitors, a dimensionally homogeneous equation suitable for use in the present inventive method is Equation [1]:
ρ=1/(1+ψ_{I}^{-h}) [1],
wherein ρ is the dimensionless signal, ψ_{I} is the dimensionless inhibitor concentration and h is the Hill coefficient of the enzyme inhibited by the enzyme inhibitor. The dimensionally homogeneous equation can alternatively be Equation [2]:

$${ρ}_{i} = \frac{1}{1 + {( \frac{{EC}_{50}}{[ Inhibitor ]} )}^{h}} , \qquad [ 2 ]$$

wherein ρᵢ is the dimensionless signal, EC₅₀ is the concentration at which 50% of the enzyme velocity is affected (effective concentration at 50%), “[Inhibitor]” is the concentration of the enzyme inhibitor, and h is the Hill coefficient of the enzyme. The EC₅₀ can be obtained by methods known in the art (see, for example, Kuo, J. F. (1994) Protein Kinase C. Oxford, New York N.Y.; Reith, A. D. (2001) Protein Kinase Protocols. Humana Press, Totowa N.J.;), while [Inhibitor] is a known parameter in the assay. The Hill coefficient of the enzyme can be obtained by a least squares fit of equations 1 or 2 to the empirical data.

In the foregoing methods, wherein the dimensionally homogeneous equation is either Equation [1] or Equation [2], the method identifies the enzyme inhibitor having the most activity and/or the enzyme inhibitor having the least activity. Use of Equation [2] takes into account the differential effects of enzyme velocity that each of the one or more enzyme inhibitors have on the enzyme, whereas use of Equation [1] takes into account the different binding cooperativities of the one or more enzyme inhibitors. By “binding cooperativity” is meant that the binding affinity of successive inhibitor molecules to the enzyme is affected by the initial binding of inhibitor molecules. If a Hill slope is positive, the binding affinity of successive inhibitor molecules is greater compared to the initial binding of inhibitor molecules. In this case, the bound inhibitor molecules cause an increase in the binding affinity for subsequent inhibitor molecules. If a Hill slope is negative, the binding affinity of successive inhibitor molecules is lower compared to the primary binding of inhibitor molecules. In this case, the bound inhibitor molecules cause a decrease in the binding affinity for subsequent inhibitor molecules. If a Hill slope is unity, the binding of inhibitor molecules does not change the binding affinity of successive inhibitor molecules to the enzyme. Interesting phenomena occur in instances where the enzyme forms dimers and multi-mers with other enzymes and complexes with other molecules. For instance, PKC forms dimers with other PKC molecules and complexes with phosphatidyl-L-serine. In this case, the binding of an inhibitor molecule to a PKC may affect the binding affinity for inhibitor molecules to an adjacent PKC within the complex (Kuo, J. F. Protein Kinase C. Oxford, New York N.Y., (1994)).

In the case that the biological activity is activity of one or more enzymes, the dimensionally homogeneous equation can be Equation [3]:

$$ρ = \frac{{\overline{RFU}}_{sample} - {\overline{RFU}}_{MAX}}{{\overline{RFU}}_{0} - {\overline{RFU}}_{MAX}} , \qquad [ 3 ]$$

wherein ρ is the dimensionless signal, RFUₛₐₘₚₗₑ is the signal of a particular sample or enzyme assay, RFU_{MAX} is the maximum signal achieved in performing the biological assay with the one or more enzymes, and RFU₀ is the minimum signal achieved in performing the biological assay with the one or more enzymes. In this method, wherein the dimensionally homogeneous equation is Equation [3], the method identifies the enzyme having the most biological activity and/or the enzyme having the least biological activity. Although “RFU” is the abbreviation for “relative fluorescence units,” it is used herein to mean any signal measured by the assay that quantitatively correlates with the biological activity. The signal can be any signal, as discussed herein, and is not limited to being fluorescence. In the case that the biological activity is activity of one or more enzyme inducers, the dimensionally homogeneous equation can be Equation [1]:
ρ=1/(1+ψ_{I}^{-h}) [1],
wherein ρ is the dimensionless signal, ψ_{I} is the dimensionless inhibitor concentration, and h is the Hill coefficient of the enzyme induced by the enzyme inducer. The dimensionally homogeneous equation can alternatively be Equation [2′]:

$${ρ}_{i} = \frac{1}{1 + {( \frac{{EC}_{50}}{[ Inducer ]} )}^{h}} , \qquad [ {2}^{′} ]$$

ρᵢ is the dimensionless signal, EC₅₀ is the concentration at which 50% of the enzyme velocity is affected (effective concentration at 50%), “[Inducer]” is the concentration of the enzyme inducer, and h is the Hill coefficient of the enzyme. The [Inhibitor] is a known parameter of the assay. The EC₅₀ can be obtained by methods known in the art (see, for example, Kuo, Protein Kinase C, Oxford, New York, N.Y. (1994); and Reith, Protein Kinase Protocols (2001)). The Hill coefficient of the enzyme can be obtained by a least squares fit of equation 1 or 2 to the empirical data.

In the instance that the biological activity is binding activity of a ligand to a target protein, the dimensionally homogeneous equation can be Equation [4]:

$$ρ = \frac{1}{n} {\sum }_{i = 1}^{n} \frac{1}{1 + \frac{{K}_{D , i}}{C}} , \qquad [ 4 ]$$

wherein ρ is the dimensionless bound ligand concentration, n is the theoretical number of binding sites per receptor molecule, K_{d,i} is the disassociation constant (characteristic of the ligand-receptor binding affinity), and C is the concentration of the ligand. Methods of obtaining n are known in the art (see, for instance, Zhu, C. and Williams, T. E. (2000) Modeling Concurrent Binding of Multiple Molecular Species in Cell Adhesion. Biophys. J. 79 (4): 1850-7; and Eisenberg et al., Physical Chemistry With Applications To Life Science. Addison-Wesley: New York (1979)). Methods of determining K_{d,i} are known in the art (see, for instance, Mellentin-Michelotti et al., Anal Biochem. 272, 182-190 (1999); and Linnenger A. L., Nelson, D. L., and Cox, M. M. (1993) Principals of Biochemistry 2^{nd} Ed. Worth: New York, N.Y.). C is a known parameter in the assay. In this method, the method identifies the ligand having the most affinity for a target protein and/or the ligand having the least affinity for a target protein.

In the instance that the biological activity is activity of one or more ligand inhibitors, the dimensionally homogeneous equation can be Equation [5]:

$${ρ}_{b} = \frac{1}{1 + {( \frac{{EC}_{50}}{[ Inhibitor ]} )}^{h}} , \qquad [ 5 ]$$

wherein ρ_{b} is the dimensionless bound ligand concentration, EC₅₀ is the concentration at which 50% of the enzyme velocity is affected (effective concentration at 50%), “[Inhibitor]” is the concentration of the ligand inhibitor, and h is the Hill coefficient of the enzyme. In the foregoing method, wherein the dimensionally homogeneous equation is Equation [5], the method identifies the ligand inhibitor having the most activity and/or the ligand inhibitor having the least activity.

In the instance that the biological activity is activity of one or more ligand inducers, the dimensionally homogeneous equation can be Equation [5′]:

$${ρ}_{b} = \frac{1}{1 + {( \frac{{EC}_{50}}{[ Inducer ]} )}^{h}} , \qquad [ {5}^{′} ]$$

wherein ρ_{b} is the dimensionless bound ligand concentration, EC₅₀ is the concentration at which 50% of the enzyme velocity is affected (effective concentration at 50%), “[Inducer]” is the concentration of the ligand inducer, and h is the Hill coefficient of the enzyme. In the foregoing method, wherein the dimensionally homogeneous equation is Equation [5′], the method identifies the ligand inhibitor having the most activity and/or the ligand inhibitor having the least activity.

Other dimensionally homogeneous equations suitable for use in the present inventive methods are known in the art. One of ordinary skill in the art can derive such an equation through multiple steps. First, the empirical data obtained from an assay and a window of quantitation are obtained. The limits of the window depend on the assay. That is, if it is an inhibition assay, the limits would be maximum enzyme velocity and zero enzyme velocity. For a binding assay, the limits would be saturation of binding sites and zero ligand bound. The next step is to subtract the background from the assay. In an enzymatic study, the background is typically a control without any enzyme. For a binding assay, the background is typically a control without any receptor or target of the ligand. Once the background is subtracted from all of the data, the first limit of quantitation is subtracted out from all of the data. The first limit of quantitation is the data defining the region of lowest signal. This is not to be confused with the data defining the region where no enzymatic activity or no binding exists. In the proceeding example, a PKC assay is performed that is a quench assay. In this case, signal is inversely proportional to enzymatic velocity. Therefore, the region of maximum enzyme velocity defined by the data is, in fact, the region of lowest signal. Next, the difference of the limits is found by averaging the data determined from the assay that, as a control, should provide a region of highest activity and a region of lowest activity. It is important that several replicate data points be used to define the limits of quantitation in order to determine a statistically confident window. Next, the data subtracted from the first limit of quantitation is normalized with respect to the difference of the limits of quanitation. The limits define the region of highest activity as 100% and the region of lowest activity as 0% and creates a unitless parameter that is independent of the signal parameters produced by the instrument used in the assay. Next, the error of the assay most usually given by the standard deviation of replicate samples within a sample concentration is normalized with respect to the window. In this case, it is important to note that the coefficient of variation (commonly known as the standard deviation divided by the mean) does increase upon dimensional analysis. This is true because the original variation is taken within the detectable region of the instrument and not the region defined by dimensional analysis. For adequate quantitation, the detectable limits of the instrument must be larger than the detectable limits of the assay. Once the error is transformed to the dimensionless parameter, the variation increases due to the fact that the limits of the assay are smaller than the limits of the instrument. This is hard to conceptualize but can be realized if all of the data points are non-dimensionalized, and the standard deviation of the data is subsequently computed. In some cases, it is intuitive to simply normalize data to a characteristic value. For independent variables, such as inhibitor concentration and ligand concentration, Where the concentration of the analyte is known, the data can be simply normalized with a value characteristic of the data. In an inhibition study to study binding cooperativity within a class of inhibitors with varying potency, the data from several inhibition experiments are normalized with respect to the empirically found EC₅₀. This would then produce data showing the differences in binding cooperativity that would be independent of inhibitor potency. Thus, inhibitors can be more readily classified in a high throughput experiment based not only on their potency, but also on their binding characteristics.

In the present inventive methods, data are analyzed using a dimensionally homogeneous equation. As stated herein, a dimensionally homogeneous equation allows for the analysis of data, which are obtained from a biological assay, regardless of the particular day on which the biological assay was performed, regardless of the particular set of reagents and/or instruments with which the biological assay was performed, and/or regardless of the particular scale, e.g., 96-well plates, 384-well plates, and 1586-well plates, on which the biological assay was performed. For example, data obtained from a biological assay that was carried out on day 1 with 96-well plates can be directly compared to data obtained from the same biological assay that was carried out on day 2 with 384-well plates.

In this regard, the use of a dimensionally homogeneous equation in the present inventive methods can identify the most active, i.e., most potent, enzyme inhibitor of all of the enzyme inhibitors analyzed in the biological assays, as well as the least active, i.e., least potent, enzyme inhibitor of all of the enzyme inhibitors analyzed in the biological assays. The EC₅₀ of each enzyme inhibitor tested in the biological assay is determined, when the dimensionless signal parameter ρ is set to 0.5. By dimensional analysis, the EC₅₀ for an inhibitor (regardless of inhibitor type, assay time, reagents, instruments, or any other variable) always occurs at ρ=0.5. This is always true (by definition). The enzyme inhibitor having the lowest EC₅₀ is identified as the enzyme inhibitor having the most biological activity, i.e., the most potent enzyme inhibitor. The enzyme inhibitor having the highest EC₅₀ is identified as the enzyme inhibitor having the least biological activity, i.e., the least potent enzyme inhibitor. Furthermore, creating a dimensionally homogeneous equation also carries with it the ability of these data to be graphically compared. Since the data are transformed into a unitless parameter that is independent of instrument format, the data can be graphically compared to evaluate parameters like EC₅₀ and Hill slope.

In some instances, analysis of the Hill slope, which is a measure of the binding cooperativity of the enzyme inhibitor to its enzyme, is more useful than the analysis of the potency (EC₅₀) of the enzyme inhibitor. In this respect, the binding cooperativity, based on variations in the Hill slope, can be graphically evaluated. If a Hill slope is positive, the binding affinity of successive inhibitor molecules is greater compared to the initial binding of inhibitor molecules. If a Hill slope is negative, the binding affinity of successive inhibitor molecules is lower compared to the primary binding of inhibitor molecules. If a Hill slope is unity, the binding of inhibitor molecules does not change the binding affinity of successive inhibitor molecules to the enzyme. The Hill slope is entirely a characteristic of the enzyme inhibitor, and, in some cases, it is desirable to have a positive Hill slope, and, in other cases, it is desirable to have a negative Hill slope. For instance, if an enzyme inhibitor having the lowest EC₅₀ is toxic to a biological system, when the enzyme inhibitor is identified as having a negative Hill slope, the dose of the enzyme inhibitor can be adjusted to non-toxic doses. Therefore, analysis of data obtained from a biological assay quantitating the activity of one or more enzyme inhibitors allows a direct comparison of the enzyme inhibitors with respect to binding cooperativity, such that the enzyme inhibitors having a positive and/or negative Hill slope are identified.

The use of a dimensionally homogeneous equation in the present inventive methods can identify the most active, i.e., most potent, enzyme inducer of all of the enzyme inducers analyzed in the biological assays, as well as the least active, i.e., least potent, enzyme inducer of all the enzyme inducers analyzed in the biological assays. The EC₅₀ of each enzyme inducer tested in the biological assay is determined, when the dimensionless signal parameter ρ is set to 0.5. The enzyme inducer having the lowest EC₅₀ is identified as the enzyme inducer having the most biological activity, i.e., the most potent enzyme inducer. The enzyme inducer having the highest EC₅₀ is identified as the enzyme inducer having the least biological activity, i.e., the least potent enzyme inducer. Furthermore, like dimensional analysis of the enzyme inhibitors, creating a dimensionally homogeneous equation also carries with it the ability of these data to be graphically compared. Since the data are transformed into a unitless parameter that is independent of instrument format, the data can be graphically compared to evaluate parameters like EC₅₀ and Hill slope. Furthermore, like dimensional analysis of enzyme inhibitors, analysis of the enzyme inducers with respect to binding cooperativity (Hill slope), as opposed to potency (EC₅₀), can be achieved through the present inventive method.

The use of a dimensionally homogeneous equation in analyzing data obtained from one or more ligand binding assays can identify the ligand having the most biological activity, i.e., the ligand binding with highest affinity to the target. This is accomplished by determining the dissociation constant (K_{D}) for each of the ligands tested in the ligand binding assays, when the dimensionless parameter ρ is set to 0.5. By dimensional analysis, ρ=0.5 is (by definition) the dissociation constant. The ligand having the lowest K_{D} is the ligand having the highest affinity for the target. The ligand having the highest K_{D} is the ligand having the lowest binding affinity. In some cases, the ligand that has the highest affinity for the target may not be the most ideal. Creating a dimensionally homogeneous equation also carries with it the ability of these data to be graphically compared. Since the data are transformed into a unitless parameter that is independent of instrument format, the data can be graphically compared to evaluate parameters like K_{D} and B_{MAX} (saturation of ligand binding sites).

The use of a dimensionally homogeneous equation in analyzing data obtained from one or more biological assays that quantitate activity of one or more enzymes can identify the enzyme having the most activity by determining the dimensionless signal ρ for each enzyme tested in the biological assays. If the signal directly correlates with the activity of the enzymes, then the enzyme having the highest signal is the enzyme having the highest activity, and the enzyme having the lowest signal is the enzyme having the lowest activity. If the signal inversely correlates with activity of the enzymes, then the enzyme having the lowest signal is the enzyme having the highest activity, while the enzyme having the highest signal is the enzyme having the lowest activity.

The use of a dimensionally homogeneous equation in analyzing data obtained from one or more biological assays that quantitate activity of one or more ligand inhibitors can identify the ligand inhibitor having the most activity and the ligand inhibitor having the least activity by determining the EC₅₀ of each of the ligand inhibitors tested, when ρ_{b} is set to 0.5. The ligand inhibitor having the lowest EC₅₀ is the ligand inhibitor having the highest activity, whereas the ligand inhibitor having the highest EC₅₀ is the ligand inhibitor having the lowest activity.

The use of a dimensionally homogeneous equation in analyzing data obtained from one or more biological assays that quantitate activity of one or more ligand inducers can identify the ligand inducer having the most activity and the ligand inducer having the least activity by determining the EC₅₀ of each of the ligand inducers tested, when ρ_{b} is set to 0.5. The ligand inducer having the lowest EC₅₀ is the ligand inducer having the highest activity, whereas the ligand inducer having the highest EC₅₀ is the ligand inducer having the lowest activity.

As is the case with enzyme inhibitors and enzyme inducers, the data obtained from the biological assays, which quantitate the activity of one or more ligand inhibitors or ligand inducers, can be graphically expressed. Also, the data on such ligand inhibitors or ligand inducers can be analyzed with respect to binding cooperativity (Hill slope) as opposed to potency of the inhibitor or inducer (EC₅₀).

### EXAMPLES

Abbreviations

For convenience, the following abbreviations are used herein: uHTS, ultra-high throughput screening; PKC, protein kinase C; DAG, diacylglycerol; WS, Working Solution; DTT, dithiothreitol; ATP, adenosine triphosphate; RFU, relative fluorescence unit; DMSO, dimethylsulfoxide; IC₅₀, the concentration at which 50% of the enzyme velocity is inhibited (inhibition concentration at 50%); EC₅₀, the concentration at which 50% of the enzyme velocity is affected (effective concentration at 50%); PS, pseudosubstrate peptide (RFARKGSLRQKNV) N-terminous labeled with rhodamine; ρ, dimensionless signal (relative fluorescent units, RFU); h, Hill coefficient; RFUₛₐₘₚₗₑ, signal (relative fluorescent units) of sample; RFU_{MAX}, signal (relative fluorescent units) at maximum enzyme velocity; RFU₀, signal (relative fluorescent units) at zero enzyme velocity; σ, error (standard deviation); σₛₐₘₚₗₑ, error (standard deviation) of sample; ψ, dimensionless concentration (of inhibitor); FP, fluorescence polarization; mU, milliunits of enzyme (one unit of PKC is defined as the amount of enzyme that will catalyze the phosphorylation of histone with 1.0 nmol of ATP per minute at 25° C.); ELIFA, enzyme linked immunofilter assay; ELISA, enzyme linked immunosorbent assay; B, concentration of the bound ligand; C, concentration of the ligand; B_{MAX}, concentration of the ligand at saturation; K_{D}, dissociation constant; C_{R}, receptor concentration; N, average number of binding sites; σ_{B}, signal error (standard deviation) in binding assay; and n, theoretical number of binding sites per receptor molecule;

The following examples further illustrate the invention but, of course, should not be construed as in any way limiting its scope.

### Example 1

This example demonstrates that data in PKC assays can be non-dimensionalized for 96, 384, and 1536 well plates, such that the data are directly scaleable between the plate formats. This example also demonstrates that, through dimensional analysis, various classes of PKC inhibitors can be analyzed for their potency on the same scale.

Kinases are of current interest in clinical research and drug discovery. In particular, PKC functions as a critical role in signal responses to various agonists including hormones, neurotransmitters and growth factors (Kuo, Oxford: New York (1994); Izzard et al., Cancer Res. 59, 2581-2586 (1999); Shawver et al., Cancer Cell 1, 117-123 (2002); Newton, J. Biol. Chem. 270 (48), 28495-28498 (1995); Sawyers, Cancer Cell 1, 413-415 (2002); Nishizuka, Science 258, 607-612 (1992); Dekker et al., Science 19, 73-77 (1994); Bell, J. Biol. Chem. 266 (8), 4661-4664 (1991); Nishizuka, Science 258 (5082), 607-614 (1992); Schechtman et al., Met. Enzymol., 345 (37), 470-487 (2002)). These external agonists cause the intracellular level of sn-1,2-diacylglycerols (DAG) to increase (Kuo, Protein Kinase C Oxford: New York (1994); Nishizuka, Science 258, 607-612 (1992); Bell, J. Biol. Chem. 266 (8), 4661-4664 (1991)). In addition, phorbol esters and tumor promoters are strong activators of PKC. PKC is essential for the activation of platelets, neutrophils, macrophages, lymphocytes and fibroblasts that play a role in immune response (Nishizuka, Science, 258, 607-612 (1992)). In order to study PKC for potential inhibitors, a high-throughput approach must be taken. Also, showing the versatility of the assay, a method will be presented to non-dimensionalize the empirical data, such that it tells more information regardless of plate format or inhibitor type. Data seen at all scales of plate formats and inhibitor data from different classes of inhibitors are related back to dimensionless scales to assess their potency and their similar functions.

A summary of the assay formats, enzyme reaction mixture volumes, and IQ™ Working Solution volumes for assays conducted in various microwell plate formats is shown in Table 1. IQ™ Working Solution was prepared by mixing 9 volumes of IQ™ Reagent A and 1 volume of IQ™ Reagent B prior to use to provide full strength IQ™ WS (1×IQ™ WS). The full-strength working solution was further diluted with water to prepare half-strength or quarter-strength IQ™ working solution. In 96 well assays, 4× reaction volume of quarter-strength was added and in 384 and 1536 well assays, 2× reaction volume of half-strength was added.

Enzymatic assays were conducted at room temperature for 1 hour unless otherwise indicated. Reactions were carried out in microwell plates and initiated by addition of ATP or enzyme. Kinase experiments used PKC purified from rat brain. PKC assays were conducted in a final enzyme reaction mixture consisting of 20 mM HEPES, 1 mM CaCl₂, 5 mM MgCl₂, 1 mM disodium ATP, 1 mM DTT, 0.2 mg/ml phosphatidyl-L-serine, pH 7.4. PKC assays were conducted in a final enzyme reaction mixture consisting of 20 mM HEPES, 5 mM MgCl₂, 1 mM disodium ATP, 1 mM dithiothreitol, 0.1 mM cAMP, pH 7.4. The amounts of enzyme utilized reflect a common unit definition where 1 mU of enzyme is the amount of enzyme required to transfer 1 pmole phosphate per minute at 30° C.

Each enzymatic reaction mixture used a fluorophore-labeled peptide substrate as indicated; a summary of peptides utilized is shown in Table 2. Peptides were labeled on the N-terminus with a proprietary red fluorescent label. Fluorescence was measured using either a BMG PolarStar™ or BMG Optima™ fluorescent plates readers with a 560/590 excitation/emission filter set for the red-fluorophore label. Results were typically obtained by autogaining the instrument to 90% of full-scale PMT deflection using a control (no enzyme) well. Data were plotted either as observed relative fluorescence units (RFUs) or after normalization of the RFU to control wells.

Inhibitor studies using the IQ Platform with PKC/PS: IC₅₀ values for known PKC inhibitors were determined using fluorophore-labeled pseudosubstrate peptide as the substrate. Inhibitor studies were carried out in triplicate using 500 μU PKC per μl of final enzyme reaction mixture and 60 μM peptide substrate. Inhibitors, diluted in either aqueous solution for water-soluble inhibitors or in 10% DMSO for water-insoluble inhibitors, were incubated with the enzyme for 20 minutes at room temperature, after which the enzymatic reaction was initiated by addition of ATP at a final concentration of 20 μM. The enzymatic reaction was carried out for 1 hour at room temperature followed by addition of 36 μl of 0.5×IQ WS per well. Fluorescence was then measured and data were analyzed using sigmoidal nonlinear regression.

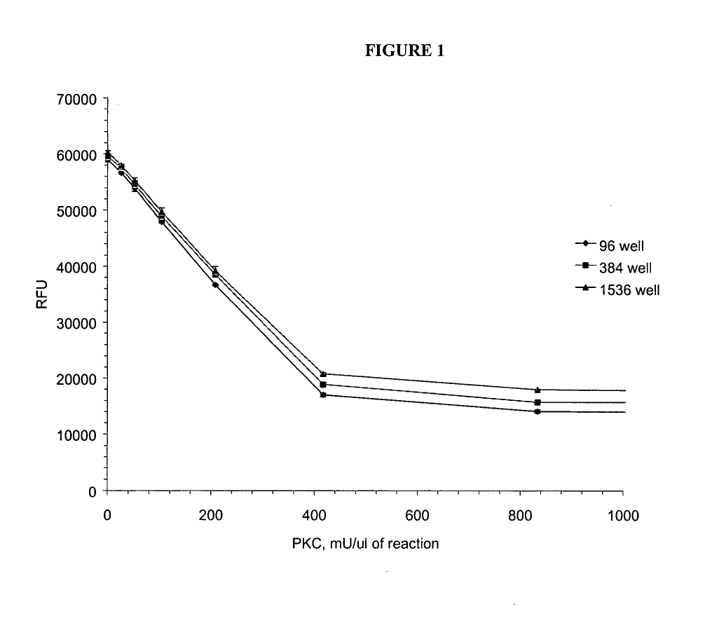

Many different assays are completed in different plate formats depending on available equipment, cost, and other factors. In recent years, the move to increase throughput and decrease reagent cost has made smaller well formats more popular. Comparing between different plate formats is necessary for validation and accurate data interpretation. In this study, different plate formats were compared. An identical reaction mixture was made for each plate format and aloquated into each plate format. The instrument was autogained for each plate on the same well containing no enzyme. FIG. 1 shows the raw RFU for each plate type.

From FIG. 1, the same reaction mixuture will give different values between different plate formats. This is the reason behind running standards and assay controls in the same plate format as the samples to be analyzed. Through dimensional analysis, this is not necessarily true. The data can be transformed to give the same numerical value, regardless of plate format. If the graph is nondimensionalized, the minimum RFU, which is obtained at the point where enzyme concentration is independent of the RFU obtained, is defined to be zero, and the negative control without enzyme, which results from only collisonal quenching by the iron, is defined to be unity. Thus, the enzyme concentration, where the enzyme velocity is at a maximum (V_{MAX}), will be zero on the quench curve, and the enzyme concentration, where there is no enzymatic activity (V=0), will be unity. Equation 1 shows this relation, such that the dimensionless group rho is defined as

$$ρ = \frac{{\overline{RFU}}_{sample} - {\overline{RFU}}_{MAX}}{{\overline{RFU}}_{0} - {\overline{RFU}}_{MAX}} \qquad ( 1 )$$

where RFU₀ is defined as the RFU without enzyme for each respective plate format and RFU_{MAX} is defined as the RFU obtained at maximum enzyme velocity (V_{MAX}). Thus, increasing enzyme concentration beyond RFU_{MAX} will have no effect on RFU, since the enzymatic velocity is at a maximum. In these assays, it is necessary to have several data points past RFU_{MAX} so that it can be accurately defined.

The error associated with the assay is non-diminsionalized as in equation 2.

$$σ = \frac{{σ}_{sample}}{{\overline{RFU}}_{0} - {\overline{RFU}}_{MAX}} \qquad ( 2 )$$

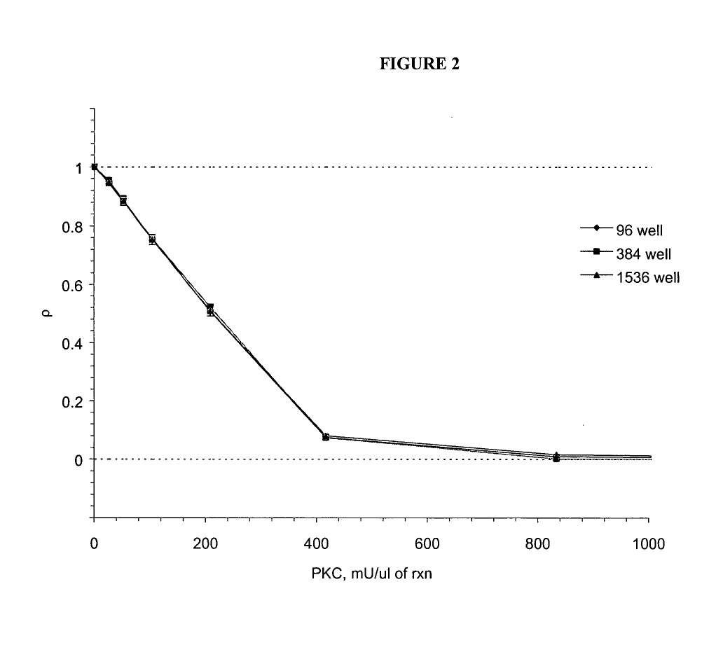

In FIG. 2, the non-dimensionalized values of each of the three plate formats are shown. Unlike FIG. 1, FIG. 2 shows that the three plate formats are directly scalable giving superimposable curves upon dimensional analysis. Thus, the data obtained for a certain quantity of enzyme will yield the same non-dimensional fluorescence in each of the three well formats shown. As a result, this allows for direct comparison of data between well formats.

Taking it a step further, various inhibitors that affect PKC activity can be compared. Depending on the class of inhibitor and the site specificity, some inhibitors affect the enzyme velocity differently than others. For example, a non-competitive inhibitor affects the V_{MAX} and a competitive inhibitor affects the K_{M}, and an uncompetitive inhibitor affects both V_{MAX} and K_{M}. In doing an inhibition experiment, it is necessary to stop the reaction, such that the reaction is still in the transient state. Otherwise, the enzyme velocity differential with dose response will not be seen. In comparing a large number of inhibitors, it is almost physically impossible to stop all of the assays at the same time in a side-by-side experiment. Even with automation, there would be a lag time that would be significant between addition of reagent to each respective inhibitor. Also, if an assay is done on a separate day, preformed on a separate instrument, or in a different plate format, it is not possible to compare the raw RFU's obtained from one experiment to another.

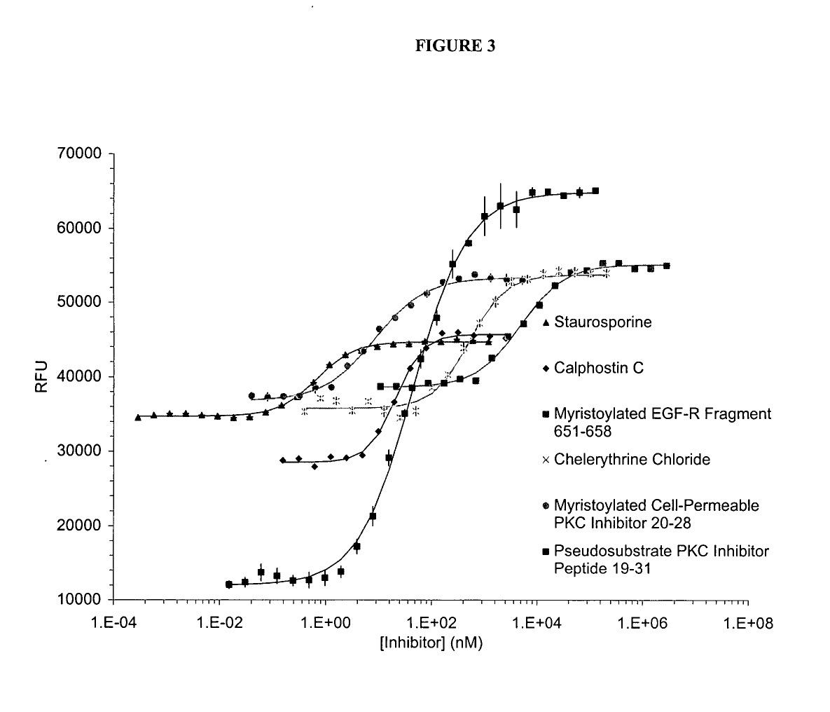

As in FIG. 3, we see the RFU of various inhibitor reactions done independently with the gain set respective to each type of inhibitor. The top and bottom baselines refer to complete inhibition and no inhibition. At complete inhibition, the enzyme velocity is essentially zero. Table 1 shows a diverse group of C kinase inhibitors that was chosen to complete this assay.

In FIG. 3, the general shape of each inhibition curve is similar; however, the baselines for the different inhibitors are drastically different. This is due to a number of factors, including the fact that the inhibitor reaction must be stopped in the transient state for an accurate differential in enzyme velocity. This is due from the fact that the velocity differential through a gradient of inhibitor concentrations must be looked at. Also, since the inhibitors were screened on different days with different gain settings, the RFU scales, themselves, are very different throughout the inhibitors screened.

We must first realize that RFU's are relative fluorescent units that represent a gradient relative to only the assay, itself. RFU's are reflective of the gain setting of the detection PMT unit of the fluorometer. Reading the same plate at a different gain setting would produce drastically different RFUs from the previous read. If an assay were preformed on a separate day, on a separate instrument, or in a different plate format, the raw RFU's would only be applicable to its respective experiment. Thus, RFU's represent a quantitative range based solely on the assay measurement system and do not reflect any true emission of product from the assay sample as in a radioactive assay.

With respect to a dimensional analysis of kinase inhibition, the top baseline of the inhibition curve can be defined as 100% inhibition (V=0) and the bottom baseline can be defined as 0% inhibition, and a dimensionless group similar to equation 1 can be formulated.

$${ρ}_{i} = \frac{{\overline{RFU}}_{sample} - {\overline{RFU}}_{0 \% inh}}{{\overline{RFU}}_{100 \% inh} - {\overline{RFU}}_{0 \% inh}} \qquad ( 3 )$$

where RFU_{100% inh} is the RFU obtained from the top baseline (100% inhibition) and RFU_{100% inh} is the RFU obtained from the bottom baseline. As in equation 1, it is necessary to have several data points at both baselines so that accurate values for RFU_{100%} and RFU_{0%} can be calculated. Like equation 1, the error associated with equation 3 is

$${σ}_{i} = \frac{{σ}_{sample}}{{\overline{RFU}}_{100 \% inh} - {\overline{RFU}}_{0 \% inh}} . \qquad ( 4 )$$

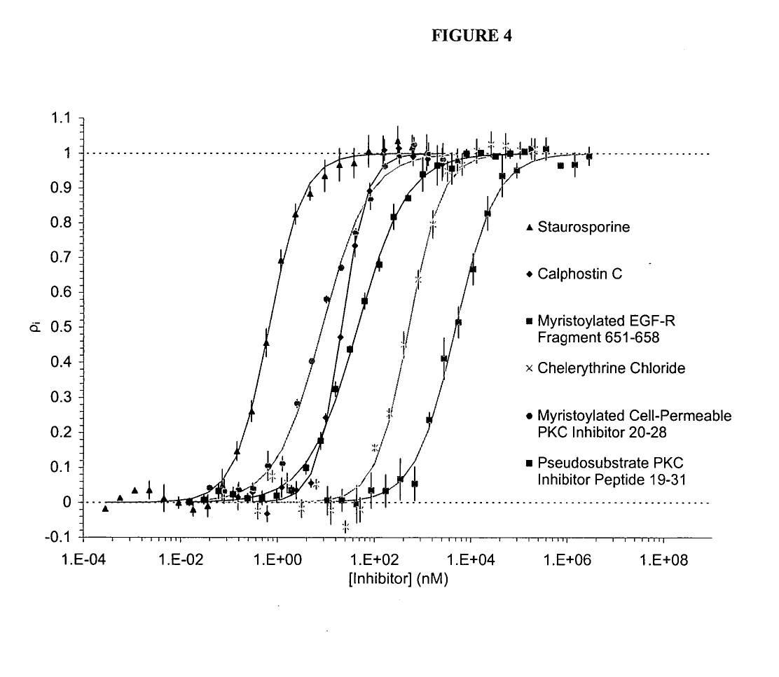

Taking the data from FIG. 3 and non-dimensionalizing it according to equations 3 and 4 results in FIG. 4. From FIG. 4, the potency of various inhibitors based on how they affect the enzyme velocity, can be observed to be different. The inhibition curves in FIGS. 3 and 4 follow a sigmodial regression given by equation 5 (Kuo, Protein Kinase C, Oxford, New York (1994)). Equation 5 represents the governing equation of a sigmodial dose response curve.

$$\overline{RFU} = {\overline{RFU}}_{0 \% inh} + \frac{{\overline{RFU}}_{100 \% inh} - {\overline{RFU}}_{0 \% inh}}{1 + {( \frac{{EC}_{50}}{[ Inhibitor ]} )}^{h}} \qquad ( 5 )$$

where h is the Hill Coefficient referring to the binding cooperativity of the inhibitor and the substrate and EC₅₀ is the concentration at which 50% inhibition takes place (effective concentration, 50%).

Upon dimensional analysis of equation 5 incorporating rho for the dimensionless RFU,

$${ρ}_{i} = \frac{1}{1 + {( \frac{{EC}_{50}}{[ Inhibitor ]} )}^{h}} . \qquad ( 6 )$$

From FIG. 4, differences between potencies of inhibitors can be observed, such that inhibitors with smaller EC50's are more potent. Another area of interest is the dose response of the intermediate region. In FIG. 4, it can be seen that the slopes of the inhibitors of the intermediate region drastically differ. In equations 5 and 6, the intermediate region slope is dependent on the Hill slope.

From FIG. 4, the differences in the slope of the intermediate region are not exactly seen. It is necessary to see the slope of the intermediate region to determine how the Hill coefficient differs between inhibitor types. To further analyze the intermediate region of the dose response curves relative to each type of inhibitor, we must define a dimensional group for the inhibitor concentration must be defined. Normalizing the inhibitor concentration to the EC₅₀ of the respective inhibitor, the dimensional group psi in equation 7 is obtained.

$${Ψ}_{i} = \frac{[ Inhibitor ]}{{EC}_{50}} \qquad ( 7 )$$

Now, using psi as the dimensionless value for inhibitor concentration, the governing equation for inhibitor dose response (equation 6) simplifies to the following relation.

$${ρ}_{i} = \frac{1}{1 + {Ψ}_{i}^{- h}} \qquad ( 8 )$$

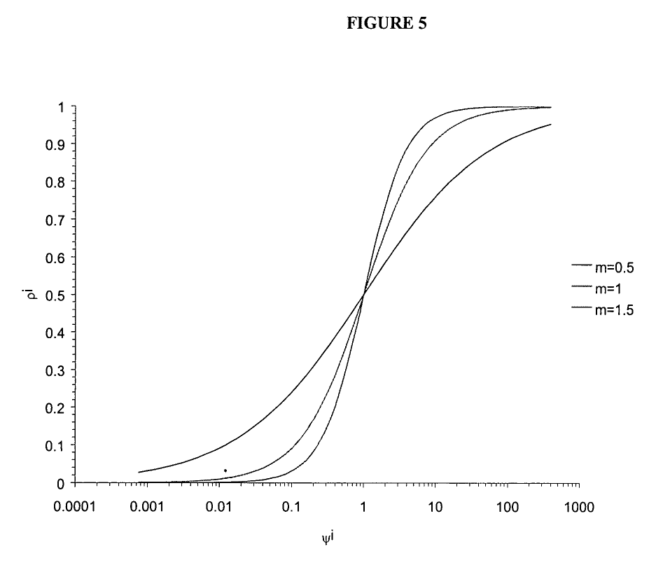

Plotting this as a model, FIG. 5 shows the different inhibition curves associated with varying Hill coefficients. This model shows that, if a difference in binding cooperativity is present, it can be seen upon dimensional analysis. This would have not been accurately represented in FIG. 3. Binding cooperativity plays an important role in how the inhibitor functions in vivo and needs to be known for an accurate dose for enzyme inhibition. Taking the data from FIG. 4 and normalizing it to the respective EC50's, the Hill slopes of each inhibitor differ. The Hill slopes for each inhibitor along with the EC₅₀'s are tabulated in Table 1.

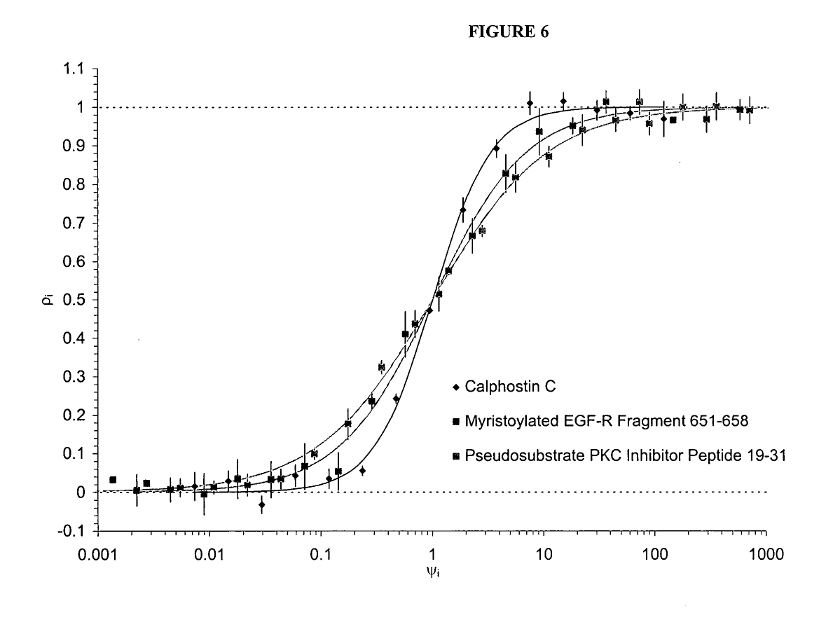

As seen in FIG. 6, the differences in the Hill slope of the intermediate region can be clearly seen. Each inhibitor can be classified based on its Hill slope, and its binding cooperativity can be more completely understood. Table 1 shows the value of the Hill slope for each inhibitor tested.

TABLE 1
IC₅₀ values and Hill slopes experimentally found with IQ assay compared to previously reported values.

| | | | | Calculated Hillslope | | |
|---|---|---|---|---|---|---|
| | | EC 50 | | | 95% confidence | Previously |
| PKC Inhibitors | Inhibitor Class | Best Fit | 95% confidence interval | Best Fit | interval | Reported IC50* |
| Staurosporine | Competitive, ATP | 0.6683 nM | 0.6090 to 0.7334 nM | 1.225 | 1.103 to 1.347 | 0.7 nM |
| | site | | | | | |
| Chelerythrine Chloride | Non-Competitive, | 524.1 nM | 459.8 to 597.4 nM | 1.273 | 1.086 to 1.460 | 700 nM |
| | ATP site | | | | | |
| Myristoylated Protein | Competitive, Peptide | 8.367 uM | 7.230 to 9.682 uM | 0.9018 | 0.7908 to 1.013 | 8.0 uM |
| Kinase C Inhibitor 20-28, | Binding Site | | | | | |
| Cell-Permeable | | | | | | |
| Myristoylated EGF-R | Competitive, Peptide | 4.837 uM | 4.199 to 5.571 uM | 1.038 | 0.9006 to 1.176 | 5.0 uM |
| Fragment (651-658) | Binding Site | | | | | |
| Pseudosubstrate PKC | Competitive, Peptide | 60.91 nM | 54.44 to 68.15 nM | 1.265 | 1.102 to 1.428 | 147 nM |
| Inhibitor Peptide 19-36 | Binding Site | | | | | |
| Pseudosubstrate PKC | Competitive, Peptide | 44.66 nM | 40.92 to 48.75 nM | 0.8462 | 0.7898 to 0.9026 | 100 nM |
| Inhibitor Peptide 19-31 | Binding Site | | | | | |
| Bisindolylmaleimide I | Competitive, ATP | 20.21 nM | 17.56 to 23.26 nM | 1.160 | 0.9937 to 1.327 | 10 nM |
| | site | | | | | |
| Calphostin C | Competitive, | 20.95 nM | 19.45 to 22.56 nM | 1.675 | 1.493 to 1.857 | 50 nM |
| | DAG/Regulatory | | | | | |
| | Domain | | | | | |

*Kuo, J. F. (1994)Protein Kinase C Oxford University Press: New York, NY.

PKC inhibitors target different parts of the active site, whether it be ATP or substrate binding, and vary in their affinity to their respective binding sites. The binding of the inhibitor to the active site is reversible based on the varying Hill slopes. Table 1 and FIG. 6 demonstrate various inhibitor classes with different binding affinities to PKC. If the Hill slope is greater than unity, this represents positive binding cooperativity. The converse is true for negative binding cooperativity. Thus, positive or negative or neutral binding cooperativity can be accurately determined with dimensional analysis.

In this study, a data management technique, which can relate data from multiple assays done independently of each other and can arrive at a common conclusion, has been shown. From these data, the data can be represented more effectively independent of assay conditions. With dimensional analysis, data performed in 96, 384, or 1536 well formats can be compared to give essentially the same curve. The fact that fluorescence quantitation requires that assignment of a value that is a relative fluorescent unit in that it is quantitative based on only the range of the instrument and not indicative of the analyte, the data can be transformed to another form that does not alter what it says, only changes the scale. Differences in fluorescence seen between plate formats are due to a number of issues, including differences in well height, surface area to volume ratio, and path length [Turconi et al., J. Biomol. Screen. 6 (5), 275-290 (2001); Winkler et al., P.N.A.S. 96, 1375-1378 (1999); and Zhang et al., J. Biomol. Screen. 4 (2), 67-73 (1999)). These differences are evident when comparing raw data from assays done in different formats. As seen here these differences are accounted for with dimensional analysis, allowing for the interpretation of data in various plate formats.

In addition, the intermediate effects with inhibitors can be compared with dimensional analysis. Different classes of inhibitors will affect PKC velocity in different respects. In order to assess the inhibition relative to each other, dimensional analysis is necessary. FIG. 4 shows the potency of different classes of inhibitors relative to each other. The dose response curves represent a gradient though a range of no enzyme velocity to maximal enzyme velocity. This gradient is dependent on the characteristics of the inhibitor, whether it is a competitive or non-competitive inhibitor, and on what binding site for which the enzyme is specific. Further non-dimensionalizing the data with respect to the EC₅₀, the binding cooperativity can be evaluated and compared with other classes of inhibitors. Comparing inhibitor binding cooperativity is necessary so that the inhibitor can be appropriately classified and properly understood relative to other inhibitors in the same class or different class. As in FIG. 4, the Hill slope will show positive or negative binding cooperativity based on the slope of the intermediate region. These differences would have gone unnoticed with the representation of the raw data as in FIG. 3.

As a result, dimensional analysis is capable of can relate results of different assay formats and scale. Also, dimensional analysis can be used to compare dose response inhibition from various classes of inhibitors. There are many other applications for dimensional analysis in the field of high-throughput screening. This data management technique will be necessary to transform experimental data so that it can be compared from a variety of platforms to reach a collective conclusion and proper representation of the assay results.

### Example 2

This example demonstrates dimensional analysis of data obtained from a binding assay.

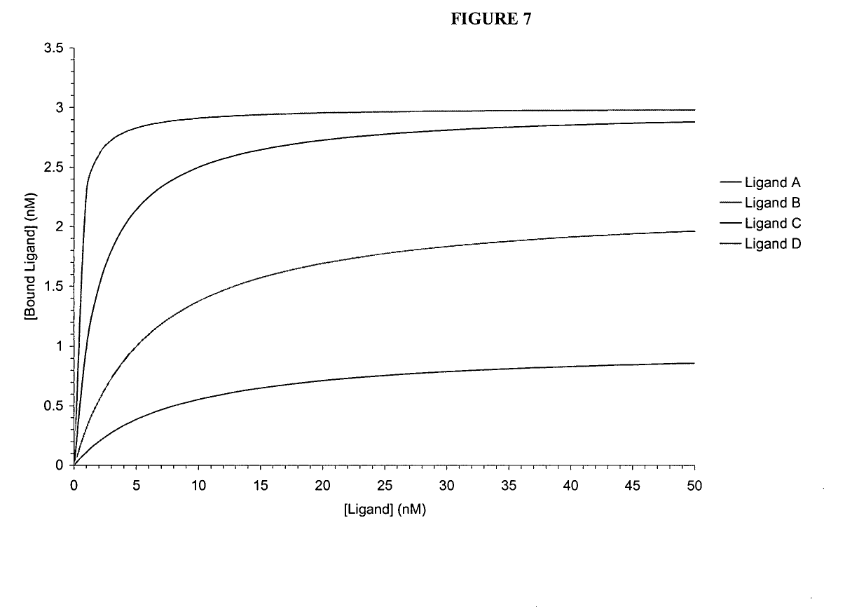

Ligand-Binding assays can be performed in a variety of formats. These formats include fluorescence polarization (Valenzano, K. J. et al., J. Biomol. Screen. 5 (6), 455-61 (2000); Klumpp et al., J. Biomol. Screen. 6 (3), 159-70 (2001); Mellentin-Michelotti et al., Anal. Biochem. 272, 182-90 (1999); and Kassack et al., J. Biomol. Screen. 7, 233-46 (2002)), ELIFA's (Nichols et al., Anal. Biochem. 257, 112-119 (1998)), ELISA's (Mahoney, Assay. Anal. Biochem. 276, 106-108, (1999)), and isotopic formats (Pissios et al., J. Steroid Biochem. and Mol. Bio. 76, 3-7(2001); Karami-Tehrani et al, Clinical Biochem. 34, 603-606 (2001)). The current governing equation describing ligand-binding is shown as equation 9 (Linnenger et al., Principals of Biochemistry 2^{nd} Ed. Worth: New York, N.Y. (1993); and Eisenberg et al., Addison-Wesley: New York (1979)).

$$B = \frac{{B}_{MAX} C}{{K}_{D} + C} \qquad ( 9 )$$

where B is the concentration of the bound ligand, C is the concentration of the ligand, B_{MAX} is the concentration of the ligand at saturation, and K_{D} is the disassociation constant. The concentration of the bound ligand is quantitated in the assay by use of a standard curve in ELISA systems or by quench curve in isotopic systems by serial dilution of known quantities of the ligand (Valenzano et al., J. Biomol. Screen. 5 (6), 455461 (2000); Klumpp et al., J. Biomol. Screen. 6 (3), 159-170 (2001); Mellentin-Michelotti et al., Anal. Biochem. 272, 182-190 (1999); Kassack et al., J. Biomol. Screen. 7, 233-246 (2002); Nichols et al., Anal. Biochem. 257, 112-119 (1998); Mahoney, Assay. Anal. Biochem. 276, 106-108 (1999); Pissios et al., Assay. J. Steroid Biochem. and Mol. Bio. 76, 3-7(2001); and Karami-Tehrani et al., Clinical Biochem. 34, 603-606 (2001)). The signal produced from the assay is dependent on the format (ELISA, FP, isotopic) as well as the ligand-receptor characteristics. The B_{MAX} is directly proportional to the number of sites available on the receptor molecule for ligand binding. FIG. 7 shows a simulation of different ligand-receptor systems, where their characteristics are noted in the table below.

TABLE 2
Ligand Binding Parameters
for Simulation shown in FIG. 1

| | B_{MAX} | K_{D} (nM) | C_{R} (nM) |
|---|---|---|---|
| Ligand A | 3 | 2 | 2 |
| Ligand B | 3.5 | 0.3 | 2 |
| Ligand C | 0.8 | 8 | 2 |
| Ligand D | 2.2 | 6 | 2 |

As seen in FIG. 7, the maximum signal is vastly different for each ligand-receptor system. This is dependent on the characteristics of the ligand-receptor binding mechanism. If B_{MAX}, which is the ligand concentration at saturation, and is normalized to the concentration of ligand binding sites in the assay, the characteristics associated with the disassociation of the ligand binding mechanism can be observed. Equation 10 identifies ρ as the normalized binding ratio. This is the number of ligands bound per total number of binding sites present in the assay.

$$ρ = \frac{B}{{C}_{R} \overline{N}} \qquad ( 10 )$$

where C_{R} is the receptor concentration in the assay and N is the average number of binding sites per receptor molecule. B_{MAX} is the total number of binding sites at saturation. Thus, B_{MAX} is the equivalent of the total number of binding sites present in the assay. Thus, the number of binding sites per receptor molecule can be represented as equation 11.

$$\overline{N} = \frac{{B}_{MAX}}{{C}_{R}} \qquad ( 11 )$$

Equation 11 is an important characteristic of a ligand-binding mechanism in that it produces an average ratio of ligands that can bind to a receptor molecule. However, taking a step further and looking at the disassociation characteristics of the mechanism, the response of the ligand-binding mechanism with the average ratio (N) can be non-dimensionalized.

Applying equations 2 and 3 to the governing equation for ligand binding, equation 9 becomes equation 12.

$$ρ = \frac{1}{1 + \frac{{K}_{D}}{C}} \qquad ( 12 )$$

The error obtained from the assay is similarly normalized as in equation 13.

$$σ = \frac{{σ}_{B}}{{C}_{R} \overline{N}} \qquad ( 13 )$$

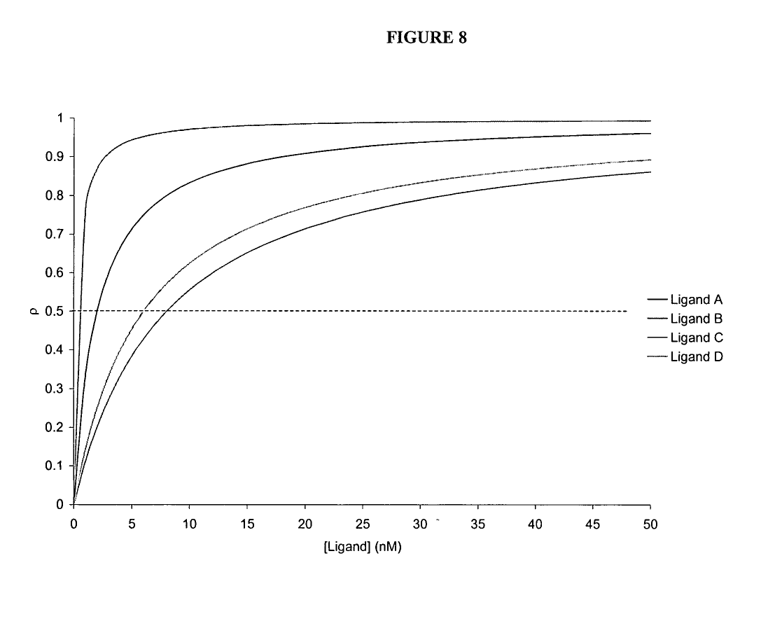

FIG. 8 shows the simulation shown in FIG. 7 normalized with respect to N. As seen in FIG. 8, all ligand-binding mechanisms reach a maximum prescribed by their average number of binding sites per molecule. From FIG. 8, it can be seen that drastic differences in the ligand binding affinity can affect the binding ratio (ρ). Ligand binding affinity is proportional to the disassociation of the ligand-receptor complex.

From this point, it has been assumed that the binding of subsequent ligands to a receptor molecule is of equal affinity to that of the binding of a primary ligand. In some systems, the binding affinity of receptor molecules changes with subsequent binding of ligands (Eisenberg et al., Addison-Wesley: New York, (1979)). For this case, the governing equation for ligand binding is defined as equation 14.

$$B = {\sum }_{i = 1}^{n} \frac{{B}_{MAX} C}{{K}_{D , i} + C} \qquad ( 14 )$$

where n is the total number of binding sites per receptor molecule (Eisenberg et al., Addison-Wesley: New York, (1979)). Applying the normalization technique to equation 14, a dimensionless governing equation shown as equation 15 is obtained.

$$ρ = \frac{1}{n} {\sum }_{i = 1}^{n} \frac{1}{1 + \frac{{K}_{D , i}}{C}} \qquad ( 15 )$$

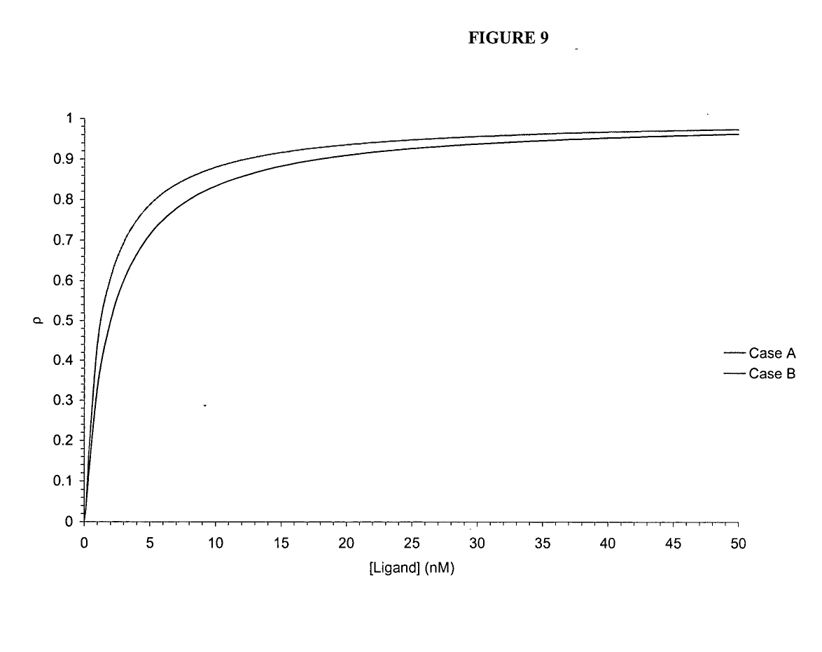

FIG. 9 shows a binary system with different binding affinities for each binding site (Case B, modeled as in equation 16) versus another binary system with the same binding affinities for each binding site (Case A, modeled as in equation 12).

$$ρ = \frac{1}{2} [ \frac{1}{1 + \frac{{K}_{D , 1}}{C}} + \frac{1}{1 + \frac{{K}_{D , 2}}{C}} ] \qquad ( 16 )$$

The K_{D} values for case B were 2 nM and 0.8 nM, respectively. The K_{D} value for case A was 2nM.

By non-dimensionalizing the governing equations for ligand-receptor binding (equations 1 and 8) with respect to the average number of binding sites per receptor molecule (N), the effects of ligand affinity can be analyzed and their characteristics are more graphically apparent. The average number of binding sites per receptor molecule (N), though a useful tool in characterizing ligand binding mechanisms (Nichols et al., Anal. Biochem. 257, 112-119 (1998)), was used as the primary normalization factor to non-dimensionalize the governing equation for receptor ligand binding. The governing equation in terms of N resulted, such that subtle differences in the binding affinity of ligand-receptor mechanisms can be found, and these differences are more graphically apparent.

### Example 3

This example demonstrates the derivation of dimensionally homogeneous equations useful for analyzing data obtained from a biological assay that quantitates activity of one or more ligand inhibitors or activity of one or more ligand inducers.

A two-step procedure is used for identifying the ligand inhibitor or ligand inducer having the most and/or least biological activity. First, a binding assay with the ligand alone is conducted to determine the K_{D}. Equation 17 shows the rigorous model encompassing receptors with multiple binding sites. The dimensionally homogeneous equation describing single binding sites is shown in equation 17.

$${ρ}_{b} = \frac{1}{1 + \frac{{K}_{D , i}}{C}} \qquad [ 17 ]$$

wherein ρ_{b} is dimensionless bound ligand concentration, K_{D,i} is the disassociation constant (characteristic of the ligand-receptor binding affinity), and C is the concentration of the ligand. Once the K_{D} of the system is known, a dose response experiment at 2×K_{D} is performed and the EC₅₀ of the inhibitor is found. The K_{D} value at 2× provides an adequate concentration of ligand to measure the highest possible range between saturation and 0% bound, yet is low enough, so that non-specific binding is minimized. The dimensionally homogeneous equation for dose response of a ligand binding system can be Equation [18]:

$${ρ}_{b} = \frac{1}{1 + {( \frac{{EC}_{50}}{[ Inhibitor ]} )}^{h}} \qquad [ 18 ]$$

wherein ρ_{b} is dimensionless bound ligand concentration, EC₅₀ is the concentration at which 50% of the enzyme velocity is affected (effective concentration at 50%), “[Inhibitor]” is the concentration of the enzyme inhibitor, and h is the Hill coefficient of the enzyme. Methods of determining EC₅₀ for a ligand binding inhibitor are known in the art (see, for instance, Anderson et al., J. Biol. Chem., 269 (29): 19081-19807 (1994); Schror et al., Biochem. Pharm., 49 (7): 921-927 (1995); and Novick et al., Hybridoma, 10 (1): 137-146 (1991)).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of analyzing data obtained from a biological assay, which method comprises:
(a) performing a biological assay, which quantitates a biological activity,
(b) obtaining data resulting from the biological assay, and
(c) analyzing the data using a dimensionally homogeneous equation,
whereupon the data obtained from the biological assay are analyzed.

2. The method of claim 1, wherein the biological activity quantitated by the biological assay is activity of one or more enzyme inhibitors.

3. The method of claim 2, wherein the dimensionally homogeneous equation is Equation [1]:
ρ=1/(1+ψ_{I}^{-h}) [1],
and the method identifies the enzyme inhibitor having the most biological activity and/or the enzyme inhibitor having the least biological activity.

4. The method of claim 2, wherein the dimensionally homogeneous equation is Equation [2]:
$${ρ}_{i} = \frac{1}{1 + {( \frac{{EC}_{50}}{[ Inhibitor ]} )}^{h}} , \qquad [ 2 ]$$
and the method identifies the enzyme inhibitor having the most biological activity and/or the enzyme inhibitor having the least biological activity.

5. The method of claim 1, wherein the biological activity quantitated by the biological assay is activity of one or more enzymes.

6. The method of claim 5, wherein the dimensionally homogeneous equation is Equation [3]:
$$ρ = \frac{{\overline{RFU}}_{sample} - {\overline{RFU}}_{MAX}}{{\overline{RFU}}_{o} - {\overline{RFU}}_{MAX}} , \qquad [ 3 ]$$
and the method identifies the enzyme having the most activity and/or the enzyme having the least activity.

7. The method of claim 1, wherein the biological activity quantitated by the biological assay is activity of one or more enzyme inducers.

8. The method of claim 7, wherein the dimensionally homogeneous equation is Equation [1]:
ρ=1/(1+ψ_{I}^{-h}) [1],
and the method identifies the enzyme inducer having the most biological activity and/or the enzyme inducer having the least biological activity.

9. The method of claim 7, wherein the dimensionally homogeneous equation is Equation [2′]:
$${ρ}_{i} = \frac{1}{1 + {( \frac{{EC}_{50}}{[ Inducer ]} )}^{h}} , \qquad [ {2}^{′} ]$$
and the method identifies the enzyme inducer,having the most biological activity and/or the enzyme inducer having the least biological activity.

10. The method of claim 1, wherein the biological activity quantitated by the biological assay is binding activity of a ligand to a target protein.

11. The method of claim 10, wherein the dimensionally homogeneous equation is Equation [4]:
$$ρ = \frac{1}{n} {\sum }_{i = 1}^{n} \frac{1}{1 + \frac{{K}_{D , i}}{C}} , \qquad [ 4 ]$$
and the method identifies the ligand having the most affinity for a target protein and/or the ligand having the least affinity for a target protein.

12. The method of claim 1, wherein the biological activity quantitated by the biological assay is activity of one or more ligand inhibitors.

13. The method of claim 12, wherein the dimensionally homogeneous equation is Equation [5]:
$${ρ}_{b} = \frac{1}{1 + {( \frac{{EC}_{50}}{[ Inhibitor ]} )}^{h}} \qquad [ 5 ]$$
and the method identifies the ligand inhibitor having the most activity and/or the ligand inhibitor having the least activity.

14. The method of claim 1, wherein the biological activity quantitated by the biological assay is activity of one or more ligand inducers.

15. The method of claim 14, wherein the dimensionally homogeneous equation is Equation [5′]:
$${ρ}_{b} = \frac{1}{1 + {( \frac{{EC}_{50}}{[ Inducer ]} )}^{h}} \qquad [ {5}^{′} ]$$
and the method identifies the ligand inducer having the most activity and/or the ligand inducer having the least activity.
